(19) United States
(12) Patent Application Publication
(10) Pub. No.: US 2007/0078479 A1
(43) Pub. Date: Apr. 5, 2007

(54) Self-expanding vaso-occlusive devices with regulated expansion

(75) Inventors: Bronislava Belenkaya, Campbell, CA (US); Victoria Carr-Brendel, Pleasanton, CA (US)

Correspondence Address: ROBINS & PASTERNAK, 1731 EMBARCADERO ROAD, SUITE 230, PALO ALTO, CA, 94303, US

(73) Assignee: Boston Scientific Scimed, Inc.

(21) Appl. No.: 11/242,952

(22) Filed: Oct. 4, 2005

(51) Int. Cl. A61M 29/00

(52) U.S. Cl. 606/200

(57) ABSTRACT

This is a device for occluding a space within the body. In particular, the device comprises a self-expanding material and an element that regulates the extent of the expansion of the self-expanding material. The devices may be placed in a desired site within a mammal.

### FIELD OF THE INVENTION

Compositions and methods for repair of aneurysms are described. In particular, vaso-occlusive devices comprising an expandable material are disclosed, as are methods of making and using these devices.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings which may be dimensioned to engage the walls of the vessels. (See, e.g., U.S. Pat. No. 4,994,069 to Ritchart et al.) Other less stiff helically coiled devices have been described, as well as those involving woven braids. See, e.g., U.S. Pat. No. 6,299,627.

U.S. Pat. No. 5,354,295 and its parent, U.S. Pat. No. 5,122,136, both to Guglielmi et al., describe an electrolytically detachable embolic device. Vaso-occlusive coils having little or no inherent secondary shape have also been described. For instance, co-owned U.S. Pat. Nos. 5,690,666; 5,826,587; and 6,458,119 by Berenstein et al., describes coils having little or no shape after introduction into the vascular space. U.S. Pat. No. 5,382,259 describes non-expanding braids covering a primary coil structure.

Vaso-occlusive devices comprising one or more coatings have also been described. U.S. Pat. No. 6,280,457 discloses vaso-occlusive devices that include biodegradable coatings.

Still another approach to the embolization of an abnormal vascular site is the injection into the site of a hydrogel, such as poly(2-hydroxyethyl methacrylate) (“pHEMA” or “PHEMA”); or a polyvinyl alcohol foam (“PAF”). See, e.g., Horak et al., “Hydrogels in Endovascular Embolization. II. Clinical Use of Spherical Particles”, Biomaterials, Vol. 7, pp. 467-470 (November, 1986); Rao et al., “Hydrolysed Microspheres from Cross-Linked Polymethyl Methacrylate”, J. Neuroradiol., Vol. 18, pp. 61-69 (1991); Latchaw et al., “Polyvinyl Foam Embolization of Vascular and Neoplastic Lesions of the Head, Neck, and Spine”, Radiology, Vol. 131, pp. 669-679 (June, 1979).

U.S. Pat. No. 5,258,042 to Mehta et al. describes formulation of hydrogel materials into a preformed implant or plug that is installed in the vascular site by means such as a microcatheter. These types of plugs or implants are primarily designed for obstructing blood flow through a tubular vessel or the neck of an aneurysm, and they are not easily adapted for precise implantation within a sac-shaped vascular structure, such as an aneurysm, so as to fill substantially the entire volume of the structure.

U.S. Pat. No. 6,299,619 to Greene et al. describes an embolization device comprising one or more expansible, hydrophilic embolizing elements non-releasably carried on a filamentous carrier at spaced intervals along the length of the carrier, where the expansile elements expand upon application of saline.

U.S. Pat. No. 6,723,108 to Jones et al. discloses an expansible foam sleeve disposed around an elongated vaso-occlusive coil. However, expansion of the foam is not controlled and, as such, unpredictable swelling of the foam sleeve may lead to aneurysm rupture, parent artery stenosis or thromboembolic complications. In addition, uncontrolled long-term expansion may also create additional difficulties in coil delivery and repositioning.

Thus, there remains a need for vaso-occlusive devices comprising self-expanding materials exhibiting controlled expansion characteristics.

### SUMMARY OF THE INVENTION

Thus, this invention includes novel occlusive compositions as well as methods of using and making these compositions. In one aspect, the invention includes a vaso-occlusive device comprising an inner member; a self-expanding material, wherein the self-expanding material partially or fully surrounds the inner member; and an expansion-regulating element that controls expansion of the self-expanding material. In certain embodiments, the self-expanding material comprises a hydrogel. In other embodiments, the self-expanding material is biodegradable (e.g., comprises one or more polyesters, polyanhydrides, polysaccharides, or polyaminoacids).

In any of the devices described herein, the expansion-regulating element may comprise one or more metals and/or one or more polymers. In certain embodiments, the expansion-regulating element comprises one or more self-regulating, self-expanding hydrogels. The expansion-regulating element may cover all or some of the expanding material and can be, for example, a net-like structure, a braid, a sleeve, a coil, or the like.

Furthermore, in any of the devices described herein, the inner member may have a linear primary configuration prior to deployment and a secondary three-dimensional configuration after deployment. In certain embodiments, the inner member comprises a metal, for example, nickel, titanium, platinum, palladium, rhodium, gold, tungsten, iridium, stainless steel and alloys (e.g., super-elastic metal alloy) or combinations thereof such as nitinol.

In addition, the devices as described herein may further comprise one or more additional components. In certain embodiments, the additional component is bioactive. In other embodiments, the additional component causes a delay or deceleration of the expansion of the self-expanding material.

In yet another aspect, the invention includes a method of occluding a body cavity comprising introducing a vaso-occlusive device as described herein into the body cavity. In certain embodiments, the body cavity is an aneurysm.

Any of the devices described herein may further comprise a severable junction detachably which may be connected to a pusher element. The detachment junction can be positioned anywhere on the device, for example at one or both ends of the device. In certain embodiments, the severable junction(s) are, an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof.

In another aspect, a method of occluding a body cavity is described, the method comprising introducing a vaso-occlusive device as described herein into the body cavity. In certain embodiments, the body cavity is an aneurysm.

These and other embodiments of the subject invention will readily occur to those of skill in the art in light of the disclosure herein.

### BRIEF DESCRIPTION OF THE FIGURES

FIG. 1 is side, partial cross-section view of an exemplary embodiment according to the present invention. The self-expanding material is shown in its unexpanded configuration.

FIG. 2 is a side, partial cross-section view of the exemplary device shown in FIG. 1 and depicts the self-expanding material in its expanded configuration.

It is to be understood that the drawings depict only exemplary embodiments and are not to be considered limiting in scope.

### DESCRIPTION OF THE INVENTION

Occlusive (e.g., embolic) compositions are described. The compositions described herein find use in vascular and neurovascular indications and are particularly useful in treating aneurysms, for example small-diameter, curved or otherwise difficult to access vasculature, for example aneurysms, such as cerebral aneurysms. Methods of making and using these vaso-occlusive elements also form aspects of this invention.

All documents (publications, patents and patent applications) cited herein, whether above or below, are hereby incorporated by reference in their entireties.

It must be noted that, as used in this specification and the appended claims, the singular forms “a”, “an”, and “the” include plural referents unless the content clearly dictates otherwise. Thus, for example, reference to a device comprising “a self-expanding material” includes devices comprising of two or more materials.

The devices described herein comprise a self-expanding material covered by an additional expandable element, the outer expandable element having a fixed maximum deformation. Thus, the outer expandable element regulates expansion of the self-expanding material to prevent or decrease undesirable overexpansion.

A number of self-expanding materials can be used in the devices described herein, including but not limited to hydrogel materials Hydrogel polymers include but are not limited to, polyamino acids, polysaccharides, polyacrylic acid, polyamines, polyethylene glycols, poly(lysine)s, polyvinylalcohols (e.g., PVA), naturally occurring polymers, synthetic polymers, (including natural or synthetic collagens and natural or synthetic polysaccharides such as hyaluronic acid) and/or combinations of any of the above.

Thus, the hydrogel of the present invention may include one or more polymer components, where the polymer is naturally occurring or synthetic, or a mixture of the foregoing. A hydrogel in accordance with the invention, may be formed, for example, from organic gels and inorganic gels.

Organic gels from which the hydrogel of the invention can be selected include, by way of example and not by way of limitation, gels formed from polysaccharides and mucopolysaccharides including, but not limited to hyaluronates, pectins, agarose, alginate; chitosan, chitosan derivatives such as chitosan modified with fructose, galactose and/or carboxy alkyl celluloses, including but not limited to carboxymethyl cellulose; partially oxidized cellulose; gels formed from proteins such as collagen, gelatin, albumin, fibronectin, fibrin, poly(lysine)s or poly or copolypeptides; and gels formed from synthetic biodegradable polymers such polyphosphazenes, polyphosphoesters, polyanhydrides, polyethylene oxides, polyvinyl alcohols, polyethylene oxide-co-polypropyleneoxide block copolymers, polylactides, polyglycolide, polycaprolactone, poly(3-hydroxy-butyric acid), polyvinyl alcohols, PEG, dextran, alginic acid and sodium alginate and others such as described in U.S. Pat. No. 4,526,938 to Chirchill, et al.; gels formed from other hydroxy acids; and/or gels formed from other biologically degradable polymers that are non-toxic or are present as metabolites in the body. Inorganic gels from which the hydrogel of the invention can be selected include, by way of example and not by way of limitation, silicones, alumina, and ferric oxide.

Although hydrogel materials have been used in aneurysms, both alone and with coils, the expansion of these materials is not controlled and/or predictable. In contrast, expansion of the self-expanding materials (e.g., hydrogels) described herein is regulated by one or more additional regulating components.

In certain embodiments, the self-expanding material is biodegradable. By “biodegradable” or “bioabsorbable” is meant that the material is capable of being broken down especially into innocuous products over a period of time, ranging from days to weeks to months or even years. By “water-soluble” is meant that the molecules of the material are capable of dissolving in water. Thus, biodegradable materials include water-soluble biomaterials. Non-limiting examples of self-expanding biodegradable materials include PEO-PLA or PLA-PGA-PEO block copolymers (see, e.g., Younes et al. (1987) J. Biomed. Mater Res 21(11):1301-1306; Younes et al. (1988) Biomater Artif Cells Artif Organs. 16(4):705-19).

The self-expanding materials described herein are advantageously used in combination with other vaso-occlusive devices, for example the GDC-type vaso-occlusive coils described above (see, e.g., U.S. Pat. Nos. 6,723,112; 6,663,607; 6,602,269; 6,544,163; 6,287,318; 6,280,457 and 5,749,894). Preferably, the self-expanding material surrounds some or all of the additional vaso-occlusive device.

The additional outer expandable component that controls expansion can be fully or partially biodegradable, or, alternatively, may be permanent. Thus, virtually any expandable material can be used, including, but not limited to metals and polymers. The expansion-regulating component may be configured in any way so long as the configuration serves to limit expansion of the self-expanding material. Non-limiting examples of suitable configurations include nets, coils, ribbons, films, sutures, braids, sleeves, sheaths or other structures that are highly expandable to a maximum deformation, at which maximum deformation they serve to restrain the self-expanding material from over-expansion. See, e.g., co-owned U.S. Ser. No. 10/873,982, titled “Expanding Vaso-Occlusive Coil,” filed Jun. 21, 2004, incorporated herein by reference in its entirety. In addition, or as an alternative, the self-regulating component may include an expanding material whose expansion is self-regulated and, as such, regulates expansion of the underlying expanding material. These self-regulating expansive materials may also have any configuration suitable for restraining expansion of the self-expanding member, including, but not limited to, the configurations described above.

Depicted in the Figures are exemplary embodiments of the present invention. Although the optional inner member is depicted as a coil, it will be appreciated that this is for purposes of illustration only and that the inner member can be of other shapes, for example different shaped coils, stents, mandrels, wires, filters or the like.

Likewise, it will be appreciated that the outer expansion-regulating component, although depicted as a net-like structure in the Figures, may be any other mechanical and/or self-regulating expandable material. See, e.g., co-owned U.S. patent application, Serial No. Unassigned, titled “Self-Expanding Vaso-Occlusive Devices with Self-Controlled Expansion,” filed even date herewith. For instance, the outer-expansion-regulating component may be an outer coil shaped structure, a sleeve (cylindrical structure), a braid structure or any other structure that regulates (controls) expansion of the self-expanding material. The expansion-regulating material may be positioned over most or all of the expanding material. Furthermore, the outer expansion-regulating component may comprise one or more metals, polymers or combinations of metals and polymers.

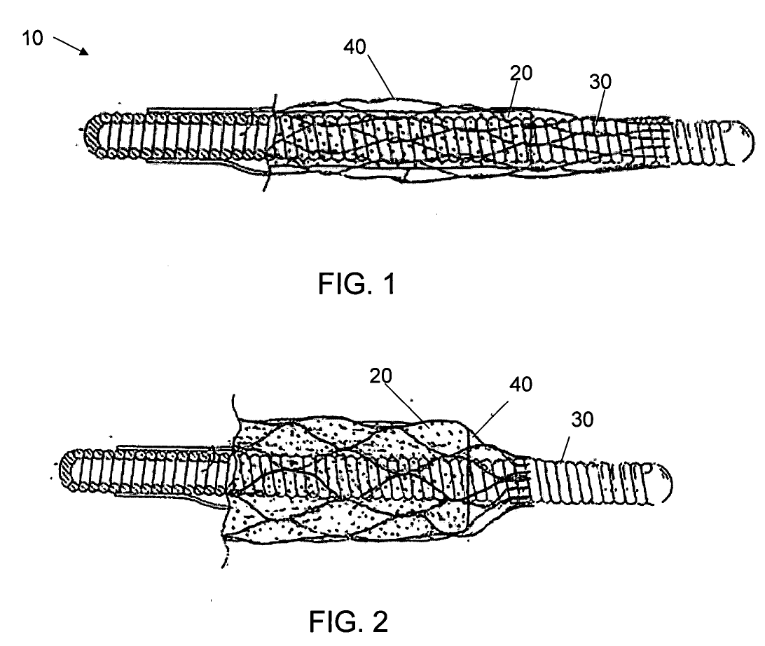

FIG. 1 depicts an exemplary embodiment of a vaso-occlusive device described herein. The device as a whole is generally designated (10). The self-expanding material (20) surrounds a helical shaped coil (30). Surrounding the self-expanding material (20) and part of the inner coil (30) is a net-like structure (40).

FIG. 2 depicts how the net-like structure (40) controls expansion of the self-expanding material (20).

As can be seen in the Figures, the expansion-regulating element can be designed to control expansion of the self-expanding material to any desired degree. For example, in the embodiments shown in FIGS. 1 and 2, the net element that controls expansion of the self-expanding material can be made to expand only a certain amount, thereby limiting overexpansion of the self-expanding material.

As noted above, the devices described herein or one or more of the components of these devices (e.g., inner member, self-expanding material, outer expansion-regulating material) described herein may assume a variety of configurations including, but not limited to, braids, coil, stents (e.g., self-expanding stents) and combinations of these. Preferably, the devices (e.g., an inner coil component) are deployed in a primary linear configuration and assume a three-dimensional configuration upon deployment. For example, the devices may form a coil configuration or may have a substantially random space-filling relaxed configuration upon deployment.

Thus, although depicted in the Figures as a coil (e.g., platinum coil), the inner member may be of a variety of shapes or configuration including, but not limited to, braids, wires, knits, woven structures, tubes (e.g., perforated or slotted tubes), injection-molded devices and the like. See, e.g., U.S. Pat. No. 6,533,801 and International Patent Publication WO 02/096273.

In certain embodiments, the inner member is a braided structure comprising one or more metals or metal alloys, for example, Platinum Group metals, especially platinum, rhodium, palladium, rhenium, as well as tungsten, gold, silver, tantalum, stainless steel and alloys of these metals. Preferably, the inner member comprises a material that maintains its shape despite being subjected to high stress, for example, “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.” The inner member may also comprise a shape memory polymer such as those described in International Publication WO 03/51444.

In certain preferred embodiments, the inner member is a platinum coil. The inner member may also change shape upon release from the restraining member, for example change from a constrained linear form to a relaxed, three-dimensional configuration upon deployment.

FIG. 2 depicts a cross-section view of the device shown in FIG. 1 during deployment from a catheter (35). Inner coil (30) is surrounded by self-expanding material (20). Self-expanding material (20) expands in a self-regulated manner after extrusion from the catheter (35).

Further, any of the devices described herein may further comprise a detachment junction, which is severable. The detachment junction may be connected to a pusher element, such as a pusher wire. The detachment junction can be positioned anywhere on the device, for example at one or both ends of the optional inner member. In certain embodiments, the inner member may be removed after deployment.

The severable junction(s) may be detached in a variety of ways, for example using an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof. Furthermore, the detachment mechanism may be hydraulic, for example the pusher wire may be cannulated, for example to allow for saline injection through the pusher wire to push off the coil.

The devices described herein may also comprise additional components, such as co-solvents, plasticizers, coalescing solvents, materials selected or designed to slow or delay the expansion of the self-expanding material (e.g., low molecular weight additives such as sucrose, inorganic salts or alcohols; or coatings based on biodegradable non-swelling polymers (PLGA, PLLA, PLCL or mixture thereof or others), bioactive agents, antimicrobial agents, thrombogenic agents, antithrombogenic agents (e.g., heparin), thrombus-stabilizing agents, antibiotics, pigments, radiopacifiers and/or ion conductors which may be coated using any suitable method or may be incorporated into the element(s) during production. See, e.g., co-owned U.S. patent application Ser. No. 10/745,911, U.S. Pat. No. 6,585,754 and WO 02/051460, incorporated by reference in their entireties herein. The bioactive materials can be coated onto or incorporated into one or more components of the device (e.g., inner coil member, self-expanding material or outer element) and/or can be placed in the vessel prior to, concurrently or after placement of one or more devices as described herein.

As noted elsewhere, the location of the device is preferably visible using fluoroscopy. A highly preferred method is to ensure that at least some of the elements (e.g., small pore non-degradable member and/or inner member) making up the device are provided with significant radio-visibility via the placement of a radio-opaque covering on these elements. A metallic coating of a metal having comparatively more visibility, during fluoroscopic use, than stainless steel is preferred. Such metals are well known but include gold and members of the Platinum Group described above.

One of more of the elements may also be secured to each other at one or more locations. For example, to the extent that various elements are thermoplastic, they may be melted or fused to other elements of the devices. Alternatively, they may be glued or otherwise fastened. Furthermore, the various elements may be secured to each other in one or more locations. Generally, if the expansion-regulating element is not biodegradable (i.e., is permanent), the self-expanding material does not need to be fixed to the inner member.

Methods of Use

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an embolus and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the vaso-occlusive device at the distal end, is advanced through the catheter.

Once the selected site has been reached, the vaso-occlusive device is extruded, for example by loading onto a pusher wire. Upon extrusion, the expansion-regulating element and self-expanding material expand until the expansion-regulating element reaches its maximum deformation, expansion of the self-expanding material is likewise limited and overexpansion prevented (FIG. 2).

Preferably, the vaso-occlusive device is loaded onto the pusher wire via a mechanically or electrolytically cleavable junction (e.g., a GDC-type junction that can be severed by application of heat, electrolysis, electrodynamic activation or other means). Additionally, the vaso-occlusive device can be designed to include multiple detachment points, as described in co-owned U.S. Pat. Nos. 6,623,493 and 6,533,801 and International Patent publication WO 02/45596. They are held in place by gravity, shape, size, volume, magnetic field or combinations thereof.

It will also be apparent that the operator can remove or reposition (distally or proximally) the device. For instance, the operator may choose to insert a device as described herein, before detachment, move the pusher wire to place the device in the desired location.

Modifications of the procedure and vaso-occlusive devices described above, and the methods of using them in keeping with this invention will be apparent to those having skill in this mechanical and surgical art. These variations are intended to be within the scope of the claims that follow.

What is claimed is:

1. A vaso-occlusive device comprising an inner member;
a self-expanding material, wherein the self-expanding material partially or fully surrounds the inner member; and
an expansion-regulating element that controls expansion of the self-expanding material.

2. The device of claim 1, wherein the self-expanding material comprises a hydrogel.

3. The device of claim 1, wherein the self-expanding material is biodegradable.

4. The device of claim 3, wherein the material comprises one or more polyesters, polyanhydrides, polysaccharides, or polyaminoacids.

5. The device of claim 1, wherein the expansion-regulating element comprises one or more metals.

6. The device of claim 1, wherein the expansion-regulating element comprises one or more polymers.

7. The device of claim 6, comprising one or more self-regulating, self-expanding hydrogels.

8. The device of claim 1, wherein the inner member has a linear primary configuration prior to deployment and a secondary three-dimensional configuration after deployment.

9. The device of claim 1, wherein the inner member comprises a metal.

10. The device of claim 9, wherein the metal is selected from the group consisting of nickel, titanium, platinum, gold, tungsten, iridium and alloys or combinations thereof.

11. The device of claim 10, wherein the metal is nitinol or platinum.

12. The device of claim 1, wherein the inner member comprises a coil comprising a metal selected from the group consisting of platinum, palladium, rhodium, gold, tungsten and alloys thereof.

13. The device of claim 1, wherein the inner member comprises a coil comprising a stainless steel or super-elastic metal alloy.

14. The device of claim 1, wherein the device further comprises a detachment junction.

15. The device of claim 14, wherein the detachment junction comprises an electrolytically detachable end adapted to detach from a pusher by imposition of a current on the pusher.

16. The device of claim 1, further comprising an additional component.

17. The device of claim 16, wherein the additional component is bioactive.

18. The device of claim 16, wherein the additional component causes a delay or deceleration of the expansion of the self-expanding material.

19. A method of occluding a body cavity comprising introducing a vaso-occlusive device according to claim 1 into the body cavity.

20. The method of claim 19, wherein the body cavity is an aneurysm.
